(19) United States
(12) Patent Application Publication
(10) Pub. No.: US 2020/0229681 A1
(43) Pub. Date: Jul. 23, 2020

(54) ENDOSCOPE

(71) Applicant: OLYMPUS CORPORATION, Tokyo (JP)

(72) Inventor: Xiongwei WANG, Tokyo (JP)

(73) Assignee: OLYMPUS CORPORATION, Tokyo (JP)

(21) Appl. No.: 16/842,875

(22) Filed: Apr. 8, 2020

Related U.S. Application Data
(63) Continuation of application No. PCT/JP2018/028708, filed on Jul. 31, 2018.

(30) Foreign Application Priority Data
Oct. 11, 2017 (JP) 2017-197536

(51) Int. Cl. A61B 1/005; A61B 1/01

(52) U.S. Cl. CPC A61B 1/0052; A61B 1/01; A61B 1/0057

(57) ABSTRACT

An endoscope includes an insertion portion, a ball, a controller, a wire, one end of which is connected to a bending portion and the other end of which is connected to a rotating body, and a groove formed so as to wind up, around the rotating body along with rotation, an intermediate portion of the wire corresponding to a direction of the wire in which the rotating body rotates.

### CROSS REFERENCE TO RELATED APPLICATION

This application is a continuation application of PCT/JP2018/028708 filed on Jul. 31, 2018 and claims benefit of Japanese Application No. 2017497536 filed in Japan on Oct. 11, 2017, the entire contents of which are incorporated herein by this reference.

### BACKGROUND OF INVENTION

### 1. Field of the Invention

The present invention relates to an endoscope equipped with an endoscope wire pulling apparatus provided with a wire, one end of which is connected to a bending portion, which is a pulled member, and the other end of which is connected to a rotating body of an operation unit.

### 2. Description of the Related Art

In recent years, endoscopes have been widely used, for example, in a medical field and an industrial field. A configuration is well known, in which, for example, a bending portion freely bendable in a plurality of directions is provided on a distal end side in an insertion portion of an endoscope.

The bending portion not only improves progression performance of the insertion portion in a bent portion in a subject or object but also makes variable an observation direction of an observation optical system provided at a distal end portion located closer to the distal end side than the bending portion in the insertion portion.

For example, one or two pairs of wires, that is, two or four wires respectively, Which are pulled members and one end of which is fixed to the bending portion, are inserted into the insertion portion of the endoscope and an operation portion of the endoscope connected to a proximal end of the insertion portion.

A configuration is well known in which any one of the four wires is pulled by an angle knob mechanism, which is an endoscope wire pulling apparatus provided in the operation portion of the endoscope.

More specifically, the angle knob mechanism has a configuration in which any one of two up-down wires wound around an up-down sprocket configured to rotate along with an up-down bending operation knob to which a rotating shaft of the up-down bending operation knob, which is an operation unit, is connected is pulled as the up-down bending operation knob rotates, any one of two left-right wires wound around a left-right sprocket configured to rotate along with a left-right bending operation knob to which a rotating shaft of a left-right bending operation knob, which is an operation unit, is connected is pulled as the left-right bending operation knob rotates, and the bending portion is thereby freely bendable in any one of four, up, down left and right directions.

Note that the angle knob mechanism also has a well-known configuration in which the bending portion is bent in any one of two up and down directions or in any one of two, left and right directions by pulling any one of the two wires wound around one bending operation knob, one rotating shaft and one sprocket.

Japanese Patent Application Laid-Open Publication No. 2017-23470 discloses a configuration in which any one of four wires is pulled by a joystick mechanism, which is an endoscope wire pulling apparatus provided in an operation portion of an endoscope to achieve intuitive bending operation of the bending portion by an operator.

More specifically, in the joystick mechanism, the end on the operation portion side of each of four, up, down, left and right wires is connected to each end portion of a cross of a cross-shaped suspension frame of a bending operation lever, which is an operation unit. The joystick mechanism has a configuration in which the bending operation lever is tilted in any one of the four, up, down, left and right directions, any one of the four wires is thereby pulled and the bending portion is freely bent in any one of the four, up, down, left and right directions.

Note that in the joystick mechanism, the ends on the operation portion side of the two wires are connected to the respective end portions of the linear suspension frame of the bending operation lever. A configuration is also well known in which the bending operation lever is tilted in two, up and down or left and right directions, any one of the two wires is thereby pulled and the bending portion is bent in any one of the two, up and down directions or in any one of the two, left and right directions.

### SUMMARY OF THE INVENTION

An endoscope according to an aspect of the present invention includes an insertion portion inserted into a subject or object and including a bending portion, one rotating body disposed on a proximal end side of the insertion portion, including a rotation center and held rotatably around the rotation center in at least three directions, a controller provided in the rotating body to allow an operator to rotate the rotating body in the at least three directions, at least three wires including one end, another end and an intermediate portion between the one end and the other end, with the bending portion connected to the one end and the rotating body connected to the other end, and wire guides provided so as to correspond to the at least three directions along the at least three wires with respect to the rotating body and formed so as to wind up, around the rotating body along with the rotation, the intermediate portions of the at least three wires corresponding to the directions in which the rotating body rotates.

An endoscope according to another aspect of the present invention includes an insertion portion inserted into a subject or object and including a bending portion, a ball made up of one spherical body or part of the spherical body disposed on a proximal end side of the insertion portion, including a rotation center and rotatably held around the rotation center, a controller provided on the ball to allow an operator to rotate the ball, a wire including one end, another end and an intermediate portion between the one end and the other end, with the bending portion connected to the one end and the ball connected to the other end, and a wire guide provided so as to correspond to a direction along the wire with respect to the ball and formed so as to wind up, around the ball along with the rotation, the intermediate portion of the wire corresponding to the direction in which the ball rotates.

### BRIEF DESCRIPTION OF THE DRAWINGS

FIG. 1 is a perspective view of an endoscope according to a first embodiment;

FIG. 2 is a diagram schematically illustrating a configuration of a spherical joystick mechanism provided inside the endoscope in FIG. 1;

FIG. 3 is an enlarged perspective view of the operation unit in FIG. 2 when a rotating body has rotated in one direction together with a wire;

FIG. 4 is a top view illustrating the operation unit in FIG. 3 viewed from a direction IV in FIG. 3;

FIG. 5 is a top view illustrating a non-rotating state of the rotating body in the operation unit in FIG. 4;

FIG. 6 is a perspective view illustrating a modification in which a shallow ball receiving groove is formed in the operation unit in FIG. 3;

FIG. 7 is a perspective view illustrating an operation unit in a spherical joystick mechanism of an endoscope according to a second embodiment when the rotating body has rotated in one direction together with a wire;

FIG. 8 is a top view illustrating the operation unit in FIG. 7 viewed from a direction VIII in FIG. 7;

FIG. 9 is a top view illustrating a non-rotating state of the rotating body in the operation unit in FIG. 8; and

FIG. 10 is a cross-sectional view of the operation unit along a line X-X in FIG. 8.

### DETAILED DESCRIPTION OF THE PREFERRED EMBODIMENT(S)

Hereinafter, embodiments of the present invention will he described with reference to the accompanying drawings. Note that the drawings are schematic ones and a relationship between thickness and width of each member, and thickness ratios among the respective members or the like are different from the actual ones. It goes without saying that there are parts, dimensional relationships and ratios of which differ among the drawings.

### First Embodiment

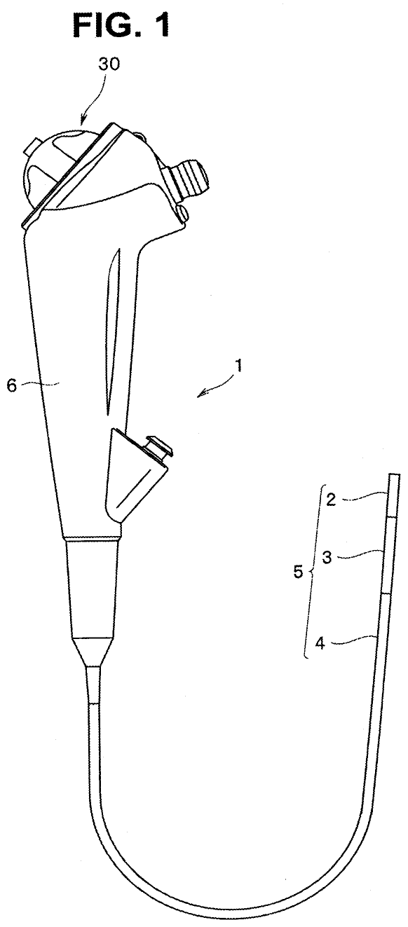

FIG. 1 is a perspective view of an endoscope according to the present embodiment.

As shown in FIG. 1, main parts of an endoscope 1 are constituted by an insertion portion 5 inserted into a subject or object and an operation portion 6 connected on a proximal end side of the insertion portion 5.

Note that a universal cord (not shown) extends from the operation portion 6 and the endoscope 1 is electrically connected to external apparatuses such as a control apparatus and an illumination apparatus via a connector (not shown) provided at an extending end of the universal cord.

The insertion portion 5 is constructed of a distal end portion 2, a bending portion 3, which is a member to be pulled and a flexible tube portion 4, in an order from a distal end side, and formed in an elongated shape.

The bending portion 3 is bent in at least three directions by an operation of an operation unit 30, which will be described late.

In this way, the bending portion 3 is intended to make variable an observation direction of an observation optical system (not shown) provided in the distal end portion 2 and improve insertability of the distal end portion 2 in the subject or object.

Note that the bending portion 3 in the present embodiment is assumed to freely bend in any one of four, up, down, left and right directions. Furthermore, the flexible tube portion 4 is connected on the proximal end side of the bending portion 3.

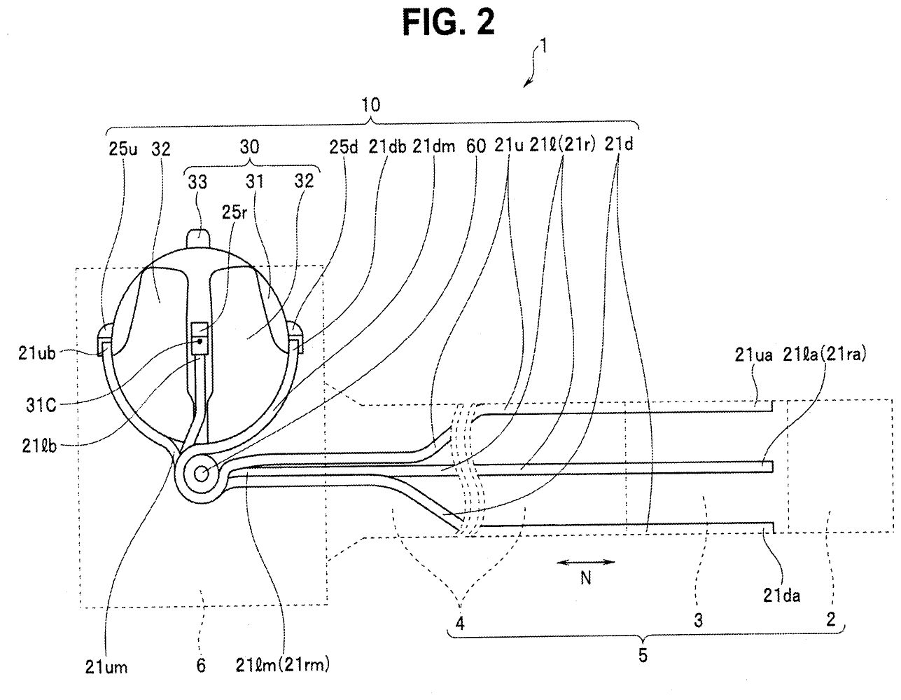

A spherical joystick mechanism 10 (see FIG. 2), which is an endoscope wire pulling apparatus configured to bend the bending portion 3 in any one of the four directions is provided in the endoscope 1. An operation unit 30 of the spherical joystick mechanism 10 is provided in the operation portion 6.

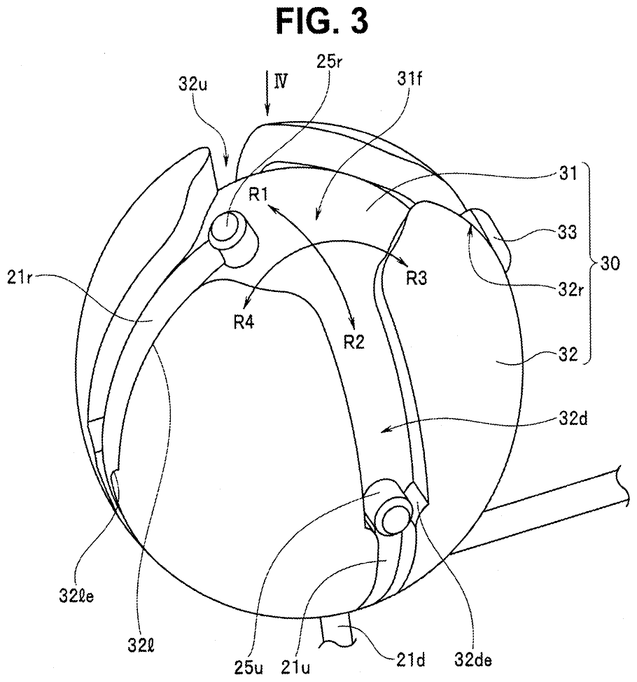

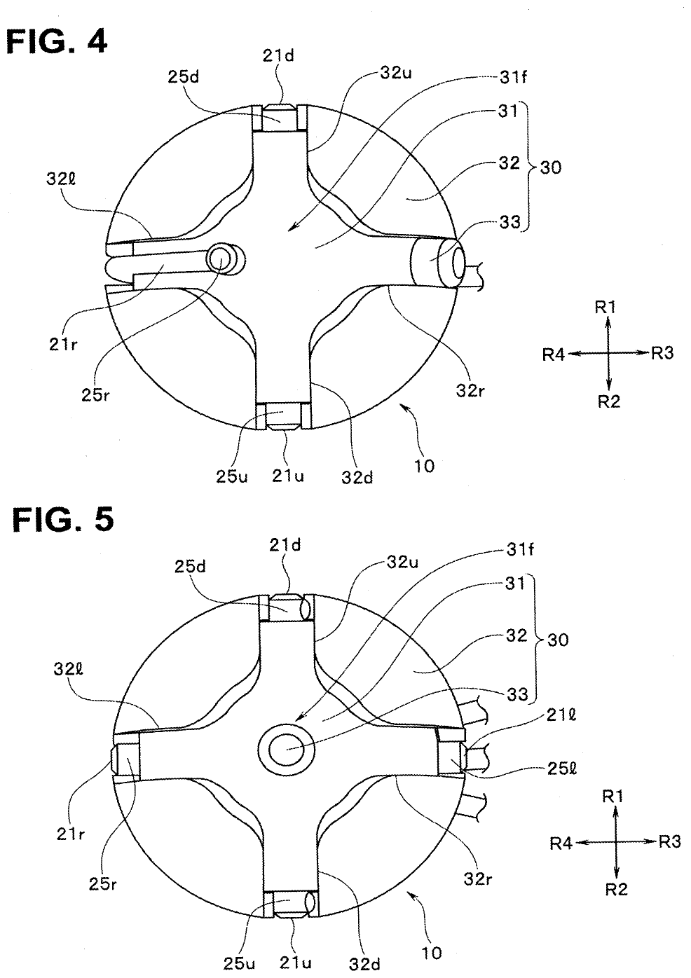

Next, a configuration of the spherical joystick mechanism 10 will be described using FIG. 2 to FIG. 5. FIG. 2 is a diagram schematically illustrating a configuration of the spherical joystick mechanism provided inside the endoscope in FIG. 1, FIG. 3 is an enlarged perspective view of the operation unit in FIG. 2 when a rotating body has rotated in one direction together with a wire, FIG. 4 is a top view illustrating the operation unit in FIG. 3 viewed from a direction IV in FIG. 3 and FIG. 5 is a top view illustrating a non-rotating state of the rotating body in the operation unit in FIG. 4.

In the following description, an end on the bending portion side of the wires 21u, 21d, 21d, 21r and 21l is referred to as one end, and an end on the operation portion side is referred to as the other end.

As shown in FIG. 2 to FIG. 5, main parts of the spherical joystick mechanism 10 are constituted by the operation unit 30 provided in the operation portion 6, wires 21u, 21d, 21r and 21l inserted into the insertion portion 5 and the operation portion 6, wire receivers 25u, 25d. 25r and 25l, which are wire fixing portions provided at respective other ends 21ub, 21db, 21rb and 21ib (the other end 21rb is not shown) of the wires 21u, 21d, 21r and 21l and grooves 32u, 32d, 32r and 32l, which are both wire movement restricting portions and wire guide portions.

Main parts of the operation unit 30 are constituted by a ball 31, which is a rotating body, a ball receiver 32, which is a holding member and a controller 33.

Note that except the controller 33, the operation unit 30 is preferably provided by being covered with a watertight cover with respect to the operation portion 6. However, the operation unit 30 may be provided exposed to outside in order to make the operation unit 30 disposable in the operation portion 6.

The ball 31 is constructed of a spherical body or part of the spherical body. The ball 31 has a rotation center 31C, which is a center of the spherical body, and is held in the ball receiver 32 so as to be rotatable in four directions (R1, R2, R3, R4) around the rotation center 31C in the present embodiment.

The controller 33, which is rotatable in four directions (R1, R2, R3, R4) together with the ball 31, in other words, configured to rotate the ball 31 in the four directions, is fixed to the surface 31f of the ball 31.

Furthermore, on the surface 31f of the ball 31, wire receivers 25u to 25l are provided at positions equidistant from the controller 33 along the rotating directions (R1, R2, R3, R4) in the four directions of the ball 31 at which the respective ends 21ub to 21lb of the wires 21u to 21l are separately fixed. In other words, the respective other ends 21ub to 21lb of the wires 21u to 21l are connected to the ball 31.

Respective one ends 21ua, 21da, 21ra, and 21la of the wires 21u to 21l are connected on the distal end sides of the bending portion 3. Respective intermediate portions 21um, 21dm, 21rm, 21dm between the respective one ends 21ua to 21l and the respective other ends 21ub to 21lb are located in the insertion portion 5 and the operation portion 6.

Furthermore, since the wire receivers 25u to 25l to which the respective other ends 21ub to 21lb are fixed are fixed at positions along the rotating directions R1 to R4 of the surface 31f as described above, the regions on the respective other end 21ub to 21lb sides of the wires 21u to 21l are wound around the surface 31f along the rotating directions R1 to R4.

The ball receiver 32 rotatably holds the ball 31 and is fixed to a frame body (not shown) provided in the operation portion 6 or an external casing of the operation portion 6.

For the ball receiver 32, in correspondence with the rotating directions R1 to R4 along the wires 21u to 21l wound around the surface 31f of the ball 31, grooves 32u to 32l are formed, configured to restrict a movement track of any one of the intermediate portions 21um to 21lm of the wires 21u to 21l corresponding to a direction in which the ball 31 rotates among the rotating directions R1 to R4 so as to wind up the intermediate portion along any one of the rotating directions R1 to R4 along with the rotation, that is, guide the intermediate portion along any one of the rotating directions R1 to R4 on the surface 31f.

Note that the groove 32u guides the intermediate portion 21dm, the groove 32d guides the intermediate portion 21um, the groove 32r guides the intermediate portion 21lm and the groove 32l guides the intermediate portion 21rm.

Note that when the controller 33 rotates the ball 31 in any one of the rotating directions R1 to R4, the controller 33 can be freely fitted into the grooves 32u to 32l.

End portions 32ue, 32de, 32re and 32le (the end portions 32ue and 32re are not shown) configured to define maximum angles of rotation of the ball 31 are formed respectively by the controller 33 coming into contact with the grooves 32u to 32l.

Note that as shown in FIG. 2, a pulley 60 may be provided in the operation portion 6, which is a wire direction changing member configured to change extending directions of the intermediate portions 21um to 21lm from the one ends 21ua to 21la to the other ends 21ub to 21lb, that is, from a longitudinal axis N of the insertion portion 5 to the rotating directions R1 to R4 respectively to restrict the movement tracks of the intermediate portions 21um to 21lm of the wires 21u to 21l along the aforementioned surface 31f and around a circumference of which the intermediate portions 21um to 21lm are wound. In other words, the intermediate portions 21um to 21lm may be wound up around the surface 31f via the pulley 60.

Note that the rest of the configuration of the spherical joystick mechanism 10 is the same as the configuration of the conventional joystick mechanism.

Next, an operation of bending the bending portion 3 in any one of four, up, down, left and right directions using the spherical joystick mechanism 10 configured above will be described.

To bend the bending portion 3 upward, the operator grasps the controller 33 first, moves (tilts) the controller 33 from the position of the controller 33 shown in FIG. 5 in the rotating direction R1 and causes the controller 33 to be fitted into the groove 32u.

As a result, the ball 31 rotates in the rotating direction R1 and the intermediate portion 21um of the wire 21u is wound up around the surface 31f in an arc shape in the rotating direction R1 with the movement track of the intermediate portion 21um of the wire 21u being restricted by the groove 32d, and the wire 21u is thereby pulled. Therefore, the bending portion 3 is bent upward. Note that the maximum bending angle of the bending portion 3 is defined by the controller 33 coming into contact with the end portion 32ue.

To bend the bending portion 3 downward, the operator grasps the controller 33, moves (tilts) the controller 33 from the position of the controller 33 shown in FIG. 5 in the rotating direction R2 and causes the controller 33 to be fitted into the groove 32d.

As a result, the ball 31 rotates in the rotating direction R2 and the intermediate portion 21dm of the wire 21d is wound up around the surface 31f in an arc shape in the rotating direction R2 with the movement track of the intermediate portion 21dm of the wire 21d being restricted by the groove 32u, and the wire 21d is thereby pulled. Therefore, the bending portion 3 is bent downward. Note that the maximum bending angle of the bending portion 3 is defined by the controller 33 corning into contact with the end portion 32de.

Furthermore, to bend the bending portion 3 rightward, the operator grasps the controller 33, moves (tilts) the controller 33 from the position of the controller 33 shown in FIG. 5 in the rotating direction R3 as shown in FIG. 3 and FIG. 4 and causes the controller 33 to be fitted into the groove 32r.

As a result, the ball 31 rotates in the rotating direction R3 and the intermediate portion 21rm of the wire 21r is wound up around the surface 31f in an arc shape in the rotating direction R3 with the movement track of the intermediate portion 21rm of the wire 21r being restricted by the groove 32l, and the wire 21r is thereby pulled. Therefore, the bending portion 3 is bent rightward. Note that the maximum bending angle of the bending portion 3 is defined by the controller 33 coming into contact with the end portion 32re.

Furthermore, to bend the bending portion 3 leftward, the operator grasps the controller 33, moves (tilts) the controller 33 from the position of the controller 33 shown in FIG. 5 in the rotating direction R4 and causes the controller 33 to be fitted into the groove 32l.

As a result, the ball 31 rotates in the rotating direction R4 and the intermediate portion 21lm of the wire 21l is wound up around the surface 31f in an arc shape in the rotating direction R4 with the movement track of the intermediate portion 21lm of the wire 21l being restricted by the groove 32r, and the wire 21l is thereby pulled. Therefore, the bending portion 3 is bent leftward. Note that the maximum bending angle of the bending portion 3 is defined by the controller 33 coming into contact with the end portion 32le.

As described above, the spherical joystick mechanism 10 is used and the bending portion 3 is bent in any one of up, down, left and right directions. Note that other operations are the same as the operations of conventional joystick mechanisms.

In this way, it has been shown in the present embodiment that the spherical joystick mechanism 10 causes the controller 33 to be fitted into any one of the grooves 32u to 32l, causes the ball 31 to rotate in any one of the rotating directions R1 to R4, winds up the intermediate portions 21um to 21lm of the wires 21u to 21l around the surface 31f of the ball 31 in an arc shape with a movement track of any one of the intermediate portions 21um to 211m of the wires 21u to 21l being restricted by any one of the grooves 32u to 32l to thereby pull any one of the wires 21u to 21l, thus causing the bending portion 3 to bend in any one of up, down, left and right directions.

According to this, compared to the configuration such as the conventional joystick mechanism where any one of four wires is pulled by tilting the bending operation lever, which is a controller, in any one of the four directions, the present embodiment adopts a configuration of winding up any one of the wires 21u to 21l around the surface 31f of the ball 31 by describing an arcuate track even when using the controller of the same diameter and with the same tilting angle and the same amount of tilting force, and so a wire pulling amount is known to increase by the order of substantially 12%.

In contrast to the joystick mechanism in the conventional product where a maximum tilting angle of the controller is on the order of substantially 60°, the present spherical joystick mechanism 10 of the same diameter has a configuration in which the controller 33 is rotated together with the ball 31, and therefore the tilting range of the controller 33 increases and the maximum tilting angle of the controller 33 can be increased to the order of substantially 80° and the wire pulling amount can also be increased by the order of substantially 50%.

Thus, it is possible to increase the wire pulling amount without providing any separate mechanism for increasing the wire pulling amount in the operation portion 6 as in the conventional art, and it is thereby possible to prevent the amount of force for operating the controller 33 to pull the wires significantly from increasing.

Furthermore, in the present embodiment, one spherical joystick mechanism 10 alone can bend the bending portion 3 in any one of up, down, left and right directions with the same wire pulling amount as the wire pulling amount of the conventional angle knob mechanism.

As described so far, it is possible to provide the endoscope 1 having the configuration in which wire pulling amounts of four wires can be increased by one mechanism while maintaining the amount of operation force of the operation unit 30 without increasing the scale of the pulling mechanism.

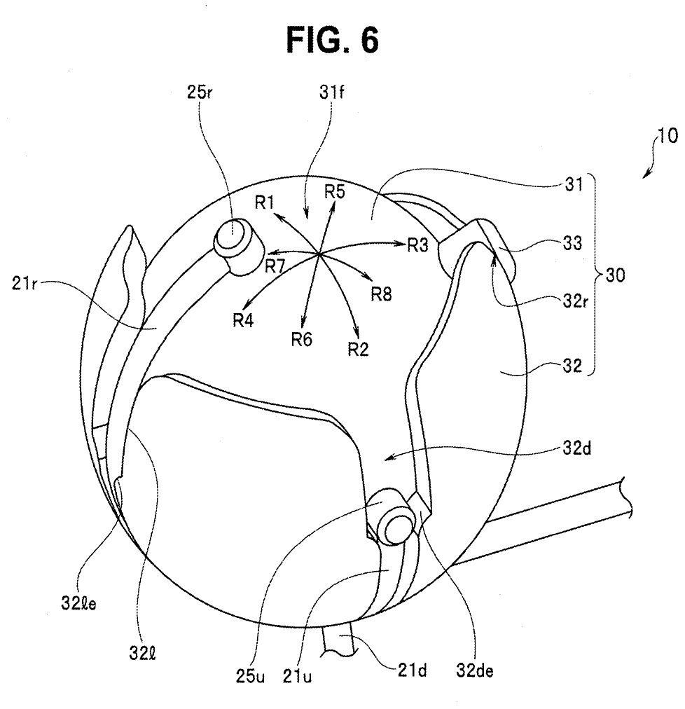

Hereinafter, modifications will be shown. FIG. 6 is a perspective view illustrating a modification in which a shallow ball receiving groove is formed in the operation unit in FIG. 3.

Although a case has been described in the aforementioned embodiment as an example where the bending portion 3 is bent in four directions, the present invention is not limited to this, and the spherical joystick mechanism 10 of the present embodiment is also applicable to cases where the bending portion 3 is bent in at least three or more directions.

In this case, the spherical joystick mechanism 10 includes a number of wires, a number of wire receivers and a number of grooves corresponding to the number of bending directions of the bending portion 3.

However, as shown in FIG. 6, by reducing depths of the grooves 32u to 32l formed in the ball receiver 32 to thereby increase the number of tilting directions of the controller 33, that is, the number of rotating directions of the ball 31 to R1 to R8, the spherical joystick mechanism 10 may have a configuration in which the bending portion 3 is bent in eight directions by pulling any one of the wire 21u and the wire 21r, the wire 21u and the wire 21l, the wire 21d and the wire 21r, and the wire 21d and the wire 211 even with the four wire receivers 25u to 25l, four wires 21u to 21l, and four grooves 32u to 32l.

### Second Embodiment

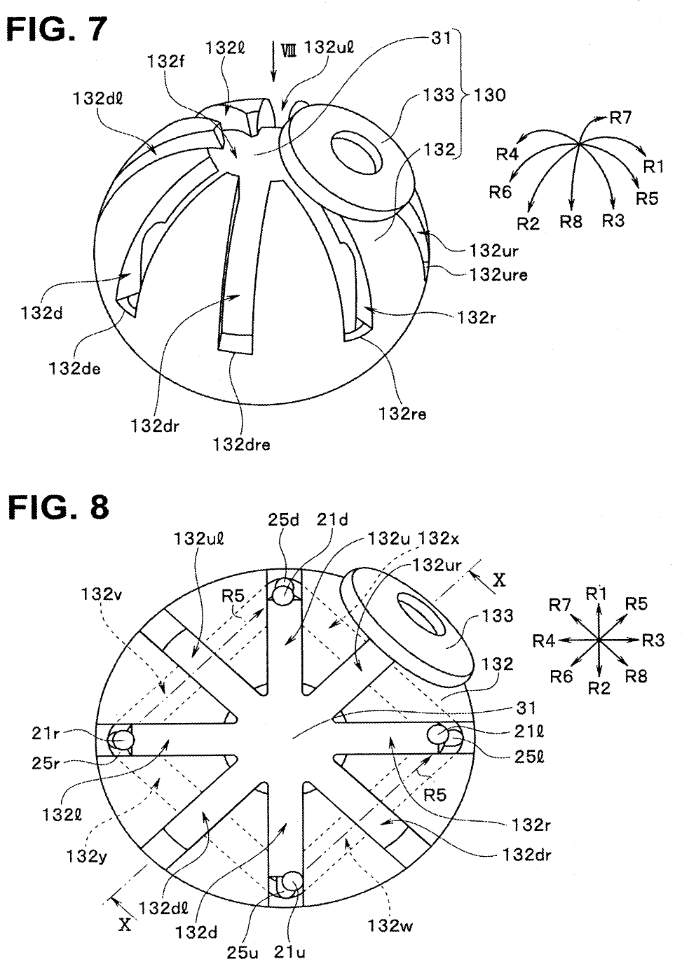

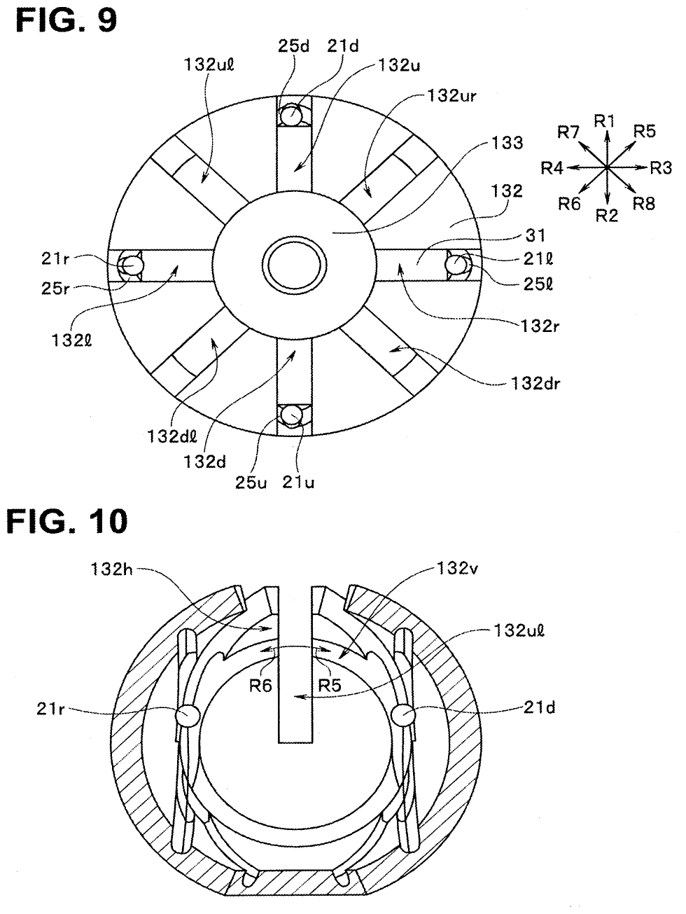

FIG. 7 is a perspective view illustrating the operation unit in a spherical joystick mechanism of an endoscope according to the present embodiment when the rotating body has rotated in one direction together with a wire, FIG. 8 is a top view illustrating the operation unit in FIG. 7 viewed from a direction VIII in FIG. 7, FIG. 9 is a top view illustrating a non-rotating state of the rotating body in the operation unit in FIG. 8 and FIG. 10 is a cross-sectional view of the operation unit along a line X-X in FIG. 8.

Compared to the spherical joystick mechanism of the endoscope of the first embodiment shown in aforementioned FIG. 1 to FIG. 5, a configuration of the spherical joystick mechanism of the endoscope of the present second embodiment is different in the number of grooves formed in the ball receiver, the number of tilting directions of the controller and the number of rotating directions of the ball according to the number of tilting directions.

Thus, only the differences will be described, and components similar to the components of the first embodiment are assigned the same reference numerals and description thereof will be omitted.

As shown in FIG. 7 to FIG. 10, main parts of the spherical joystick mechanism 10 are constituted by an operation unit 130 provided in the operation portion 6, wires 21u, 21d, 21r and 21l inserted into the insertion portion 5 and the operation portion 6, wire receivers 25u, 25d, 25r and 25l provided at the respective other ends 21ub, 21db, 21rb and 21lb of the wires 21u, 21d, 21r and 21l, and grooves 132u, 132d, 132r, 132l, 132ur, 132ul, 132dr and 132dl, which are both wire movement restricting portions and wire guide portions.

Main parts of the operation unit 130 are constituted by a ball 31, a ball receiver 132, which is a holding member and a controller 133.

Note that except the controller 133, the operation unit 130 is preferably provided by being covered with a watertight cover with respect to the operation portion 6. However, the operation unit 130 may be provided exposed to outside to make the operation unit 130 disposable in the operation portion 6.

The ball 31 is constructed of a spherical body or part of the spherical body and is rotatably held to the ball receiver 132 in eight directions (R1, R2, R3, R4, R5, R6, R7, R8) around the rotation center 31C in the present embodiment.

A shaft body (not shown) of the controller 133, which is rotatable together with the ball 31 in eight directions (R1, R2, R3, R4, R5, R6, R7, R8), in other words, causing the ball 31 to rotate in the eight directions, is fixed to the surface 31f of the ball 31.

Furthermore, since the wire receivers 25u to 25l to which the respective other ends 21ub to 21lb are fixed are fixed at positions along the rotating directions R1 to R4 of the surface 31f as in the case of the above-described first embodiment, the regions on the respective other end 21ub to 21lb sides of the wires 21u to 21l are located by being wound around the surface 31f in the rotating directions R1 to R4.

Note that in the present embodiment, as shown in FIG. 2, a pulley 60 may also be provided in the operation portion 6, which is configured to change extending directions of the intermediate portions 21um to 211m of the wires 21u to 21l from the longitudinal axis N of the insertion portion 5 to the rotating directions R1 to R4 respectively, and around a circumference of which the intermediate portions 21um to 21lm are wound.

The bail receiver 132 is intended to rotatably hold the ball 31 and is fixed to a frame body (not shown) provided in the operation portion 6 or to an external casing of the operation portion 6.

For the ball receiver 132, grooves 132u, 132d, 132r, 132l, 132v, 132w, 132x and 132y are formed, which are configured to guide any one of the intermediate portions 21um to 211m of the wires 21u to 21l corresponding to a direction in which the ball 31 rotates among the rotating directions R1 to R8 in correspondence with the rotating directions R1 to R8 to restrict movement tracks so as to wind up the wires along any one of the rotating directions R1 to R8 along with the rotation, that is, guide the wires along any one of the rotating directions R1 to RS around the surface 31f.

Note that the groove 132u guides the intermediate portion 21dm, the groove 132d guides the intermediate portion 21um, the groove 132r guides the intermediate portion 21lm and the groove 132l guides the intermediate portion 21rm.

Furthermore, as shown in FIG. 8 and FIG. 10, on an inner surface 132h of the ball receiver 132, a groove 132v is formed in a direction connecting the groove 132u and the groove 132l. A groove 132w is formed in a direction connecting the groove 132d and the groove 132r. Furthermore, a groove 132x is formed in a direction connecting the groove 132du and the groove 132r. A groove 132y is formed in a direction connecting the groove 132d and the groove 132l.

Note that the groove 132v guides the intermediate portion 21dm or the intermediate portion 21rm, and the groove 132w guides the intermediate portion 21um or the intermediate portion 211m. The groove 132x guides the intermediate portion 21dm or the intermediate portion 211m and the groove 132y guides the intermediate portion 2 turn or the intermediate portion 21rm.

Note that when the controller 133 causes the ball 31 to rotate in any one of the rotating directions R1 to R8, the controller 133 is freely fitted into the groove 132u, 132d, 132r, 132l, 132ur, 132ul, 132dr or 132dl.

The shaft body of the controller 133 comes into contact with the groove 132u, 132d, 132r, 132l, 132ur, 132ul, 132dr or 132d. As a result, end portions 132ue, 132de, 132re, 132le, 132ure, 132ule, 132dre and 132dle (none except the end portion 132re, 132de, 132ure or 132dre is shown) for defining a maximum angle of rotation. of the ball 31 are formed respectively.

Note that the rest of the configuration of the spherical joystick mechanism 10 is the same as the configuration of the first embodiment.

Next, an operation of bending the bending portion 3 in any one of up, down, left and right directions and eight directions resulting from combinations among them using the spherical joystick mechanism 10 configured above will be described.

To bend the bending portion 3 upward, the operator grasps the controller 133 first, moves (tilts) the controller 133 from the position of the controller 133 shown in FIG. 9 in the rotating direction RI and causes the controller 133 to be fitted into the groove 132u.

As a result, the ball 31 rotates in the rotating direction R1 and the intermediate portion 21um of the wire 21u is wound up around the surface 31f in an arc shape in the rotating direction R1 with the movement track of the intermediate portion 21um of the wire 21u being restricted by the groove 132d, and the wire 21u is thereby pulled.

Therefore, the bending portion 3 is bent upward. Note that the maximum bending angle of the bending portion 3 is defined by the shaft body of the controller 133 coming into contact with the end portion 132ue.

To bend the bending portion 3 downward, the operator grasps the controller 133, moves (tilts) the controller 133 from the position of the controller 133 shown in FIG. 9 in the rotating direction R2 and causes the controller 133 to be fitted into the groove 132d.

As a result, the ball 31 rotates in the rotating direction R2 and the intermediate portion 21dm of the wire 21d is wound up around the surface 31f in an arc shape in the rotating direction R2 with the movement track of the intermediate portion 21dm of the wire 21d being restricted by the groove 132u, and the wire 21d is thereby pulled.

Therefore, the bending portion 3 is bent downward. Note that the maximum bending angle of the bending portion 3 is defined by the shaft body of the controller 133 coming into contact with the end portion 132de.

Furthermore, to bend the bending portion 3 rightward, the operator grasps the controller 133, moves (tilts) the controller 133 from the position of the controller 133 shown in FIG. 9 in the rotating direction R3 and causes the controller 133 to be fitted into the groove 132r.

As a result, the ball 31 rotates in the rotating direction R3 and the intermediate portion 21rm of the wire 21r is wound up around the surface 31f in an arc shape in the rotating direction R3 with the movement track of the intermediate portion 21rm of the wire 21r being restricted by the groove 132l, and the wire 21r is thereby pulled.

Therefore, the bending portion 3 is bent rightward. Note that the maximum bending angle of the bending portion 3 is defined by the shaft body of the controller 133 coming into contact with the end portion 132re.

Furthermore, to bend the bending portion 3 leftward, the operator grasps the controller 133, moves (tilts) the controller 133 from the position of the controller 133 shown in FIG. 9 in the rotating direction R4 and causes the controller 133 to be fitted into the groove 132l.

As a result, the ball 31 rotates in the rotating direction R4 and the intermediate portion 21lm of the wire 21l is wound up around the surface 31f in an arc shape in the rotating direction R4 with the movement track of the intermediate portion 211m of the wire 21l being restricted by the groove 132r, and the wire 21l is thereby pulled.

Therefore, the bending portion 3 is bent leftward. Note that the maximum bending angle of the bending portion 3 is defined by the shaft body of the controller 133 coming into contact with the end portion 132le.

Furthermore, to bend the bending portion 3 in a composite direction of upward and rightward directions, the operator grasps the controller 133, moves (tilts) the controller 133 from the position of the controller 133 shown in FIG. 9 in the rotating direction R5 as shown in FIG. 7 and FIG. 8 and causes the controller 133 to be fitted into the groove 132ur.

As a result, the ball 31 rotates in the rotating direction R5 and the intermediate portions 21um and 21rm of the wire 21u and the wire 21r are wound up around the surface 31f in an arc shape in the rotating direction R5 with the movement tracks of the intermediate portions 21um and 21lm of the wire 21u and the wire 21r being restricted by the grooves 132v and 132w, and the wire 21u and the wire 21r are thereby pulled.

Therefore, the bending portion 3 is bent in the composite direction of upward and rightward directions. Note that the maximum bending angle of the bending portion 3 is defined by the shaft body of the controller 133 coming into contact with the end portion 132ure.

Furthermore, to bend the bending portion 3 in a composite direction of downward and leftward directions, the operator grasps the controller 133, moves (tilts) the controller 133 from the position of the controller 133 shown in FIG. 9 in the rotating direction R6 and causes the controller 133 to be fitted into the groove 132dl.

As a result, the ball 31 rotates in the rotating direction R6 and the intermediate portions 21dm and 211m of the wire 21d and the wire 21l are wound up around the surface 31f in an arc shape in the rotating direction R6 with the movement tracks of the intermediate portions 21dm and 211m of the wire 21d and the wire 21l being restricted by the grooves 132v and 132w, and the wires 21d and 21l are thereby pulled.

Therefore, the bending portion 3 is bent in the composite direction of downward and leftward directions. Note that the maximum bending angle of the bending portion 3 is defined by the shaft body of the controller 133 coming into contact with the end portion 132dle.

Furthermore, to bend the bending portion 3 in a composite direction of upward and leftward directions, the operator grasps the controller 133, moves (tilts) the controller 133 from the position of the controller 133 shown in FIG. 9 in the rotating direction R7 and causes the controller 133 to be fitted into the groove 132ul.

As a result, the ball 31 rotates in the rotating direction R7 and the intermediate portions 21um and 21lm of the wire 21u and the wire 21l are wound up around the surface 31f in an arc shape in the rotating direction R7 with the movement tracks of the intermediate portions 21um and 21lm of the wire 21u and the wire 21l being restricted by the grooves 132x and 132y, and the wires 21u and 21l are thereby pulled.

Therefore, the bending portion 3 is bent in the composite direction of upward and leftward directions. Note that the maximum bending angle of the bending portion 3 is defined by the shaft body of the controller 133 corning into contact with the end portion 132ule.

Furthermore, to bend the bending portion 3 in a composite direction of downward and rightward directions, the operator grasps the controller 133, moves (tilts) the controller 133 from the position of the controller 133 shown in FIG. 9 in the rotating direction R8 and causes the controller 133 to be fitted into the groove 132dr.

As a result, the ball 31 rotates in the rotating direction R8 and the intermediate portions 21dm and 21rm of the wire 21d and the wire 21r are wound up around the surface 31f in an arc shape in the rotating direction R8 with the movement tracks of the intermediate portions 21dm and 21rm of the wire 21d and the wire 21r being restricted by the grooves 132x and 132y, and the wires 21d and 21r are thereby pulled.

Therefore, the bending portion 3 is bent in the composite direction of downward and rightward directions. Note that the maximum bending angle of the bending portion 3 is defined by the shaft body of the controller 133 coming into contact with the end portion 132dre.

As described above, the spherical joystick mechanism 10 is used and the bending portion 3 is bent in any one of the up, down, left and right directions and a composite directions of these directions.

Note that other operations are the same as the operations of the first embodiment, and using one spherical joystick mechanism 10 in such a configuration, it is possible to cause the bending portion to bend in eight directions using further four grooves formed on the inner surface 132h in addition to the eight grooves, while obtaining effects similar to the effects of the first embodiment.

Note that although a case has been described in the aforementioned first and second embodiments where the wire guide portions are grooves formed in the hall receiver, but the present invention is not limited to this, and it goes without saying that the wire guide portions may also be grooves formed on the surface 31f of the ball 31.

What is claimed is:

1. An endoscope comprising:
an insertion portion inserted into a subject or object and comprising a bending portion;
one rotating body disposed on a proximal end side of the insertion portion, including a rotation center and held rotatably around the rotation center in at least three directions;
a controller provided on the rotating body to allow an operator to rotate the rotating body in the at least three directions;
at least three wires comprising one end, another end and an intermediate portion between the one end and the other end, with the bending portion connected to the one end and the rotating body connected to the other end; and
wire guides provided so as to correspond to the at least three directions along the at least three wires with respect to the rotating body and formed so as to wind up, around the rotating body along with the rotation, the intermediate portions of the at least three wires corresponding to the directions in which the rotating body rotates.

2. The endoscope according to claim 1, wherein
the rotating body is a spherical body or part of the spherical body, and
the rotation center is a center of the spherical body.

3. The endoscope according to claim 2, further comprising a holding member configured to rotatably hold the spherical body, wherein
the wire guides are grooves formed in the holding member and configured to guide the intermediate portions of the at least three wires along a surface of the spherical body.

4. The endoscope according to claim 3, wherein
end portions configured to define a maximum angle of rotation of the spherical body in the at least three directions are formed in the grooves.

5. The endoscope according to claim 4, wherein
the controller is formed so as to be freely fitted into the grooves, and
the end portions are formed in the holding member at at least three locations so that the controller conies into contact with the end portions.

6. The endoscope according to claim 1, wherein the intermediate portions of the at least three wires are wound up around the rotating body via a pulley configured to change extending directions of the at least three wires from the one end sides to the other end sides.

7. The endoscope according to claim 2, wherein the wire guides are at least three grooves formed on a surface of the spherical body in the at least three directions.

8. An endoscope comprising:
an insertion portion inserted into a subject or object and comprising a bending portion;
a ball made up of one spherical body or part of the spherical body disposed on a proximal end side of the insertion portion, including a rotation center and rotatably held around the rotation center;
a controller provided on the ball to allow an operator to rotate the ball;
a wire including one end, another end and an intermediate portion between the one end and the other end, with the bending portion connected to the one end and the ball connected to the other end; and
a wire guide provided so as to correspond to a direction along the wire with respect to the ball and formed so as to wind up, around the ball along with the rotation, the intermediate portion of the wire corresponding to the direction in which the ball rotates.
